(19) United States
(12) Patent Application Publication
(10) Pub. No.: US 2003/0216719 A1
(43) Pub. Date: Nov. 20, 2003

(54) Method and apparatus for treating skin using patterns of optical energy

(76) Inventors: Len Debenedictis, Palo Alto, CA (US); George Voevodkin, Tarrytown, NY (US); Michael Black, Foster City, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/367,582

(22) Filed: Feb. 14, 2003

Related U.S. Application Data
(63) Continuation-in-part of application No. 10/279,093, filed on Oct. 22, 2002.
(63) Continuation-in-part of application No. 10/278,582, filed on Oct. 23, 2002.
Application No. 10/278,582 is a continuation-in-part of application No. 10/017,287, filed on Dec. 12, 2001.
Application No. 10/278,582 is a continuation-in-part of application No. 10/020,270, filed on Dec. 12, 2001.

(51) Int. Cl. A61B 18/18

(52) U.S. Cl. 606/10

(57) ABSTRACT

A dermatological apparatus includes multiple light source and optical pathway connections. Each light source is capable of delivering an optical beam through its connected optical pathway to a targeted portion of a human skin. The dermatological apparatus also includes a control system to select and control the light sources to deliver multiple optical beams in a discontinuous pattern and a focusing element to focus the power of the delivered optical beams to multiple discrete treatment zones that are located up to 1.5 mm underneath an outer surface of the targeted portion. The discrete treatment zones have sizes in the range of 10 μm to 1000 μm.

### PRIORITY

The present application claims priority from and is a continuation-in-part of U.S. patent application Ser. No. 10/279,093, filed on Oct. 22, 2002 and entitled “Method and Apparatus for Treating Skin Using Patterns of Optical Energy” (Attorney Docket No. RTEC-008/00US), and U.S. patent application Ser. No. 10/278,582, filed on Oct. 23, 2002 and entitled “Dermatological Apparatus and Method” (Attorney Docket No. RTEC-002/00US), which claims priority from and is a continuation-in-part of U.S. patent application Ser. No. 10/017,287, filed on Dec. 12, 2001 and entitled “Multiple Laser Treatment” (prior Attorney Docket No. RLT-111), and U.S. patent application Ser. No. 10/020,270, filed on Dec. 12, 2001 and entitled “Multiple Laser Diagnostics” (prior Attorney Docket No. RLT-112), the disclosures of which are incorporated herein by reference in their entirety.

### FIELD OF THE INVENTION

The present invention relates generally to treatment of biological tissues using optical energy. More particularly, the present invention relates to methods and apparatus for treating skin using patterns of optical energy.

### BACKGROUND OF THE INVENTION

Optical energy has many useful applications for the treatment of skin and other biological tissues. For example, lasers have been used to treat dermatological conditions such as hemangiomas, port wine stains, rosacea, superficial pigmented lesions, and fine wrinkles.

Current dermatological laser methods and apparatus typically irradiate a relatively large and continuous area of a skin during treatment. However, treatment of such large area can induce an excessive degree of trauma to the skin as well as lead to the development of complications such as hypopigmentation or white spots. Furthermore, the current paradigm of treating a large area can impede normal repair processes of the skin and the flow of nutrients to the treated area, which not only can slow down healing but also may lead to necrosis and scarring. Some of the current methods and apparatus have attempted to overcome these negative effects by including a complex cooling system to cool down the skin in an attempt to reduce excessive heat development at the surface of the skin and resulting trauma to an epidermal layer of the skin. However, such cooling system adds complexity to implementation, often requires that laser power be increased, and also may not provide a desired or uniform level of cooling and trauma reduction of the skin. The combination of non-uniformity in cooling and increased laser power can put the skin at an even greater risk of damage. And, adjusting the fluence delivered by a laser, as specified by current procedures, generally provides an inadequate level of control and often leads to either over-treatment or under-treatment. Over-treatment may cause scarring, and under-treatment may result in no observable improvement in the dermatological condition being treated. Since changes may not be visible for weeks to months after treatment, there is a significant clinical problem associated with either over-treatment or under-treatment.

It is against this background that a need arose to develop the methods and apparatus described herein.

### SUMMARY OF THE INVENTION

In one particularly innovative aspect, the present invention is directed to a dermatological apparatus. In one embodiment, the dermatological apparatus may comprise a plurality of light source and optical pathway connections. Each light source in the plurality of light source and optical pathway connections is capable of delivering an optical beam through its connected optical pathway to a targeted portion of a human skin. The dermatological apparatus also may comprise a control system to select and control the light sources to deliver a plurality of optical beams in a discontinuous pattern and a focusing element to focus the power of the delivered optical beams to a plurality of discrete treatment zones that are located up to 1.5 mm underneath an outer surface of the targeted portion. The discrete treatment zones have sizes in the range of 10 μm to 1000 μm.

In another embodiment, the dermatological apparatus may comprise a plurality of light source and optical pathway connections. Each light source in the plurality of light source and optical pathway connections is capable of delivering an optical beam through its connected optical pathway to an outer portion of a human skin. The dermatological apparatus also may comprise a control system to select and control the light sources to deliver a plurality of optical beams in a discontinuous pattern and a focusing element to focus the power of the delivered optical beams to the outer portion to form a plurality of discrete holes distributed across the outer portion. The discrete holes have sizes in the range of 10 μm to 1000 μm.

In a yet another embodiment, the dermatological apparatus may comprise an optical delivery system. The optical delivery system may include an optical source and a focusing element that is optically coupled to the optical source. The optical source is configured to provide optical energy having a wavelength in the range of 400 nm to 20,000 nm, and the focusing element is configured to direct the optical energy in a discontinuous pattern to a targeted portion of a skin.

In a further embodiment, the dermatological apparatus may comprise an optical delivery system. The optical delivery system may include an optical source and a focusing element that is optically coupled to the optical source. The focusing element is configured to direct optical energy from the optical source to a targeted portion of a skin. The focusing element may include an optical lens having a numerical aperture in the range of 0.15 to 1.5, and the optical lens is configured to focus the optical energy to a dermal layer of the targeted portion.

In a yet further embodiment, the dermatological apparatus may comprise a housing sized for manipulation by a human hand, an optical source located within the housing, and a focusing element coupled to the housing. The optical source is configured to provide optical energy, and the focusing element is configured to direct the optical energy to a targeted portion of a skin such that a plurality of treatment zones within the targeted portion are exposed to the optical energy. The treatment zones are separated from one another within the targeted portion.

In a still further embodiment, the dermatological apparatus may comprise an optical delivery system. The optical delivery system is configured to direct optical energy in a pattern to a targeted portion of a skin such that a plurality of discrete treatment zones within the targeted portion are exposed to the optical energy. The discrete treatment zones have sizes in the range of 10 μm to 1000 μm.

In another particularly innovative aspect, the present invention is directed to a method of treating a human skin. In one embodiment, the method may comprise providing optical energy. The optical energy has optical parameters to produce a dermatological effect for a targeted portion of the human skin. The method also may comprise directing the optical energy to the targeted portion such that a plurality of discrete treatment zones within the targeted portion is substantially simultaneously exposed to the optical energy.

In another embodiment, the method may comprise providing optical energy and directing the optical energy to an outer portion of the human skin to form discrete holes distributed across the outer portion. The discrete holes have sizes in the range of 10 μm to 1000 μm.

### BRIEF DESCRIPTION OF THE DRAWINGS

The objectives and advantages of the present invention will be understood by reading the following detailed description in conjunction with the drawings, in which:

FIG. 1 illustrates a block diagram of a dermatological apparatus in accordance with an embodiment of the present invention;

FIG. 2 illustrates an example of a pattern of optical energy that may be directed to a targeted portion of a human skin;

FIG. 3 illustrates another example of a pattern of optical energy that may be directed to a targeted portion of a human skin;

FIG. 4 illustrates a yet another example of a pattern of optical energy that may be directed to a targeted portion of a human skin;

FIG. 5 illustrates a block diagram of a dermatological apparatus in accordance with another embodiment of the present invention;

FIG. 6 illustrates an optical delivery system in accordance with an embodiment of the present invention; and

FIG. 7 illustrates an optical delivery system in accordance with another embodiment of the present invention.

### DETAILED DESCRIPTION

Embodiments of the present invention provide an improved dermatological apparatus and method that can be used to treat skin with greater efficacy while reducing complications and healing time. In particular, embodiments of the present invention can be used to treat a wide variety of dermatological conditions such as, but not limited to, acne, birthmarks, excess hair, hemangiomas, dermal melasma, pigmented lesions, rosacea, scars, tattoos, vascular conditions, wrinkles, and so forth. While specific examples of dermatological conditions are given above, it is contemplated that embodiments of the present invention can be used to treat virtually any type of dermatological condition.

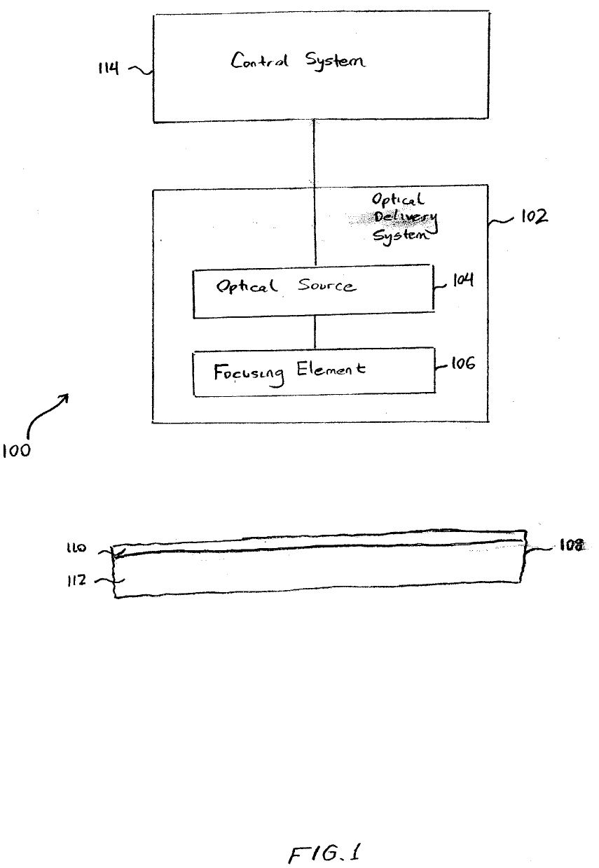

FIG. 1 illustrates a dermatological apparatus 100 in accordance with an embodiment of the present invention. The dermatological apparatus 100 includes an optical delivery system 102, which includes an optical source 104. The optical source 104 functions to provide optical energy that can be directed to a targeted portion 108 of a skin, such as a human skin. In the present embodiment, the optical source 104 provides optical energy in the form of one or more optical beams, which can be pulsed or continuous wave and coherent or incoherent.

In the present embodiment, the optical source 104 may be implemented, at least in part, using one or more light sources, such as laser light sources. For certain applications, the optical source 104 desirably includes multiple laser light sources, which can be arranged in an array, such as a one-dimensional array or a two-dimensional array. A laser light source can provide one or more optical beams having particular optical parameters, such as optical fluence, power, timing, pulse duration, inter-pulse duration, wavelength(s), and so forth, to produce a desired dermatological effect for the targeted portion 108. By way of example, a laser light source can provide an optical beam having a wavelength or range of wavelengths between approximately 400 nm and 20,000 μm, such as between approximately 600 nm and 4000 nm. For purposes of non-ablative coagulation of a dermal layer 112 of the targeted portion 108, a laser light source can provide an optical beam having a wavelength of approximately 1500 nm and an optical fluence incident on the outer surface of the skin between approximately 0.001 Joules/cm² and 10,000 Joules/cm², such as between approximately 0.1 Joules/cm² and 100 Joules/cm². For certain applications, a pulse duration of an optical beam can be approximately equal to or less than a thermal diffusion time constant associated with the targeted portion 108, which is approximately proportional to the square of the size of a focal spot within the targeted portion 108. Pulse durations that are longer than the thermal diffusion time constant can be less efficient and cause the focal spot to undesirably grow by thermal diffusion.

Examples of laser light sources include, but are not limited to, diode lasers, diode-pumped solid state lasers, Er:YAG lasers, Nd:YAG lasers, argon-ion lasers, He—Ne lasers, carbon dioxide lasers, eximer lasers, ruby lasers, and so forth. For certain embodiments, a laser light source is desirably a diode laser, such as an infrared diode laser. However, it should be recognized that the selection of a particular type of laser light source in the optical delivery system 102 is dependent on the types of dermatological conditions to be treated using the dermatological apparatus 100. The optical source 104 may include one particular type of laser light source capable of providing one wavelength or wavelength range. Alternatively, the optical source 104 may include two or more different types of laser light sources to provide a variety of different wavelengths or wavelength ranges. Optical beams from different laser light sources can be directed to the targeted portion 108 on a one-by-one basis or at the same time.

Referring to FIG. 1, the optical delivery system 102 also includes a focusing element 106 that is optically coupled to the optical source 104. The focusing element 106 functions to direct optical energy from the optical source 104 to the targeted portion 108. In the present embodiment, the focusing element 106 directs optical energy to the targeted portion 108 by focusing the power of the optical energy to one or more treatment zones within the targeted portion 108. Desirably, multiple treatment zones are simultaneously or sequentially exposed to optical energy. Multiple treatment zones can be separated from one another so as to form discrete treatment zones. Alternatively, or in conjunction, multiple treatment zones can intersect or overlap one another.

In the present embodiment, the focusing element 106 directs optical energy in a pattern, such as a discontinuous or microscopic pattern, so that one or more treatment zones are exposed to optical energy. Use of a pattern of optical energy provides greater efficacy of treatment by allowing for control of the fraction of the targeted portion 108 that is exposed to optical energy. Different patterns can provide a variety of different fractions of exposure, and a particular pattern can be selected based on the type of dermatological condition to be treated. For instance, in the case of a sensitive dermatological condition such as dermal melasma or deep pigmented lesions, use of a pattern of optical energy permits an effective level of treatment within multiple treatment zones. At the same time, by controlling the fraction of the targeted portion 108 that is exposed to optical energy, pain, immune system reaction, trauma, and other complications can be reduced. By having the treatment zones adjacent to healthy and substantially undamaged cells, healing of the targeted portion 108 is quicker, since the possibility of congestion or impairment of repair processes is reduced. Use of a pattern of optical energy also can facilitate multiple treatments that may be needed to produce a full desired effect by allowing an individual treatment to be milder and with lower risk to a patient. Furthermore, visible impressions of treatment can be reduced by using a pattern of treatment where an individual treatment zone is on the same or smaller scale than the normal visible texture or constituents of the skin itself.

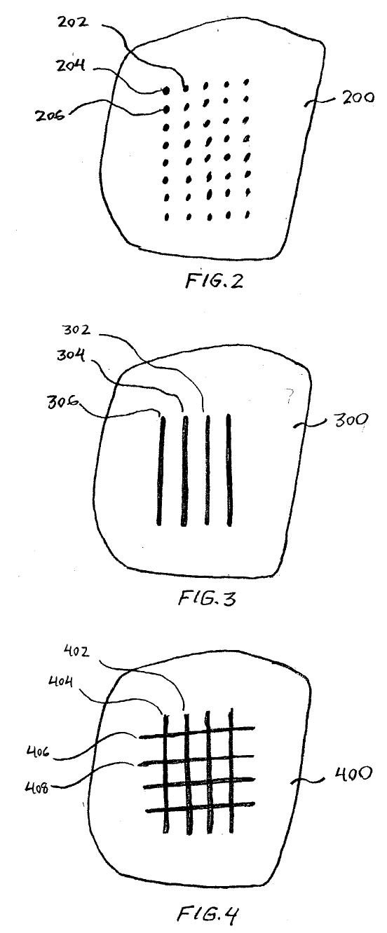

FIG. 2, FIG. 3, and FIG. 4 illustrate various examples of patterns of optical energy that may be used to treat skin. In particular, FIG. 2, FIG. 3, and FIG. 4 illustrate top views of targeted portions 200, 300, and 400, respectively, to which different patterns of optical energy are directed.

Referring to FIG. 2, optical energy is directed to the targeted portion 200 in a “dot pattern” such that multiple treatment zones, such as treatment zones 202, 204, and 206, within the targeted portion 200 are exposed to the optical energy. As seen from the top view of FIG. 2, the treatment zones are generally circular and have sizes between approximately 10 μm and 1000 μm, such as between approximately 50 μm and 500 μm. As illustrated in FIG. 2, the treatment zones are separated from one another and are distributed across the targeted portion 200 in a substantially regular manner, such as at intersection points of an imaginary grid. In the present example, two adjacent treatment zones, such as the treatment zones 202 and 204, are spaced apart by a distance between approximately 30 μm and 2000 μm, such as between approximately 100 μm and 1000 μm. The fraction of the targeted portion 200 that is exposed to optical energy can be measured using a fill factor, i.e., the fraction of the area of the targeted portion 200 that is accounted for by the treatment zones as seen from the top view of FIG. 2. In general, a fill factor can be any number in the range of 0 to 1. For certain applications, a fill factor typically ranges between approximately 0.05 and 0.95, such as between approximately 0.1 and 0.5.

Depending on the particular dermatological condition to be treated, the shapes, sizes, distribution, or fill factor associated with the treatment zones may be varied from that shown in FIG. 2 by adjusting the pattern of optical energy. The treatment zones may be formed with a variety of regular or irregular shapes, such as, by way of example and not limitation, circular, half-circular, diamond-shaped, hexagonal, multi-lobal, octagonal, oval, pentagonal, rectangular, square-shaped, star-shaped, triangular, trapezoidal, wedge-shaped, and so forth. In general, the treatment zones may have the same or different shapes or sizes. The treatment zones may be distributed across the targeted region 200 uniformly or non-uniformly and at intervals that are regularly spaced or not regularly spaced. For instance, instead of the substantially regular distribution of the treatment zones shown in FIG. 2, it is contemplated that the treatment zones may be randomly distributed across the targeted portion 200. Also, it is contemplated that the treatment zones may be distributed more sparsely at or near the edges of the targeted portion 200 to produce a “feathering effect,” which reduces the visibility of the edges and produces a more uniform result when overlapping adjoining areas of treatment. This is similar to an air brush, which achieves a blended appearance with the background and adjoining brush strokes. In addition, it is contemplated that the treatment zones may be distributed across the targeted portion 200 in an arc fashion, a circular fashion, a linear fashion, a spiral fashion, or a combination thereof.

Referring next to FIG. 3, optical energy is directed to the targeted portion 300 in a “line pattern” such that multiple treatment zones, such as treatment zones 302, 304, and 306, within the targeted portion 300 are exposed to the optical energy. As seen from the top view of FIG. 3, the treatment zones are generally elongated and have widths and lengths between approximately 10 μm and 1000 μm and between approximately 1 mm and 30 mm, respectively. The treatment zones are substantially regularly spaced apart from one another, and two adjacent treatment zones, such as the treatment zones 302 and 304, are spaced apart by a distance between approximately 30 μm and 2000 μm, such as between approximately 100 μm and 1000 μm. In a similar manner as discussed above, the fraction of the targeted portion 300 that is exposed to optical energy can be measured using a fill factor. Depending on the particular dermatological condition to be treated, the shapes, widths, lengths, distribution, or fill factor associated with the treatment zones may be varied from that shown in FIG. 3 by adjusting the pattern of optical energy. For instance, instead of the generally linear shapes of the treatment zones shown in FIG. 3, it is contemplated that one or more of the treatment zones may be shaped in an arc fashion, a circular fashion, or a spiral fashion. In general, the treatment zones may have the same or different shapes, widths, or lengths and may be distributed across the targeted portion 300 uniformly or non-uniformly and at intervals that are regularly spaced or not regularly spaced.

As illustrated in FIG. 4, optical energy is directed to the targeted portion 400 in an “intersecting line pattern” such that multiple intersecting treatment zones, such as treatment zones 402, 404, 406, and 408, within the targeted portion 400 are exposed to the optical energy. As seen from the top view of FIG. 4, the treatment zones are generally elongated and include a first set of treatment zones that intersect a second set of treatment zones at an angle. In the present example, the treatment zones may have widths, lengths, and spacings that are similar to that of the treatment zones illustrated in FIG. 3. Depending on the particular dermatological condition to be treated, the shapes, widths, lengths, distribution, or fill factor associated with the treatment zones may be varied from that shown in FIG. 4 by adjusting the pattern of optical energy. For instance, a criss-cross pattern or a honeycomb pattern of optical energy can be directed to the targeted portion 400 to vary the distribution of the treatment zones from that shown in FIG. 4.

Referring back to FIG. 1, the focusing element 106 may be implemented, at least in part, using one or more optical elements, such as mirrors, optical lenses, optical windows, and so forth, to focus the power of one or more optical beams to one or more treatment zones within the targeted portion 108. Since it is contemplated that the dermatological apparatus 100 may be used to treat a wide variety of dermatological conditions, it should be recognized that the focusing element 106 may be used to focus one or more optical beams to virtually any area or structure within the targeted portion 108, such as an epidermal layer 110 or the dermal layer 112 of the targeted portion 108.

As illustrated in FIG. 1, the dermatological apparatus 100 also includes a control system 114. The control system 114 is electronically coupled to the optical delivery system 102 via any wire or wireless transmission channel and functions to control operation of the optical delivery system 102, including the optical source 104, the focusing element 106, or both. By way of example, the control system 114 can activate one or more laser light sources of the optical source 104 as well as control a variety of optical parameters associated with an activated laser light source. As another example, the control system 114 can control the focusing element 106 to control or adjust a pattern of optical energy that is directed to the targeted portion 108. The focusing element 106 may be controlled by the control system 114 via, for instance, an electrical motor or any other device capable of positioning an optical element. While one optical delivery system 102 is shown coupled to the control system 114, it is contemplated that multiple optical delivery systems may be coupled to and controlled by the control system 114.

In the present embodiment, the control system 114 may be implemented, at least in part, using: (1) dedicated hardware or logic elements configured, for example, as a programmable gate array; (2) a typical microprocessor or central processing unit available, for example, from Intel Corp.; or (3) any typical personal computer, web appliance, or personal digital assistant product. For certain applications, the control system 114 also may include a laser driver system that interfaces with and drives the optical source 104 and a user interface to allow a user to program the control system 114.

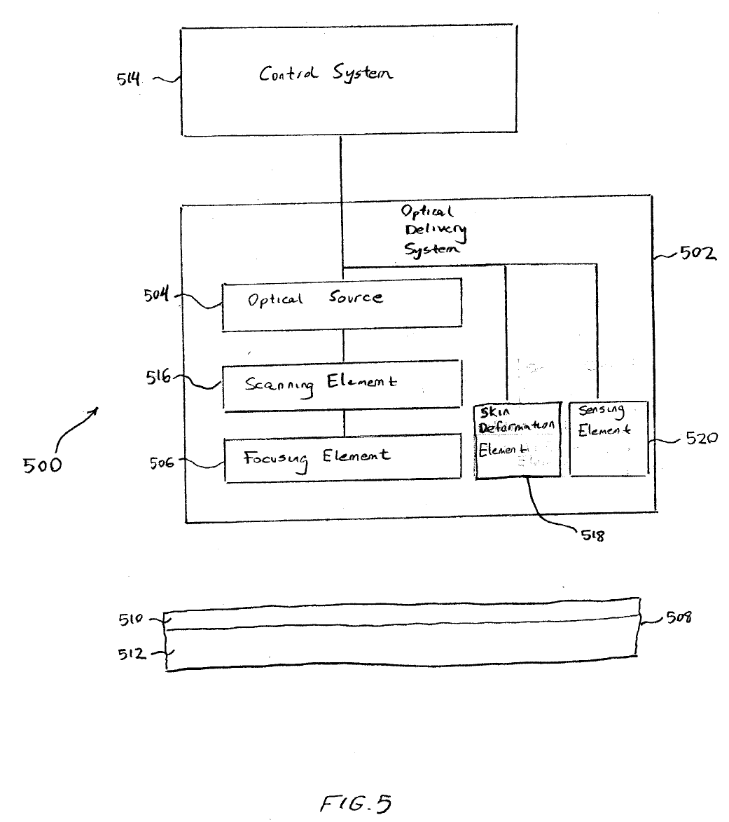

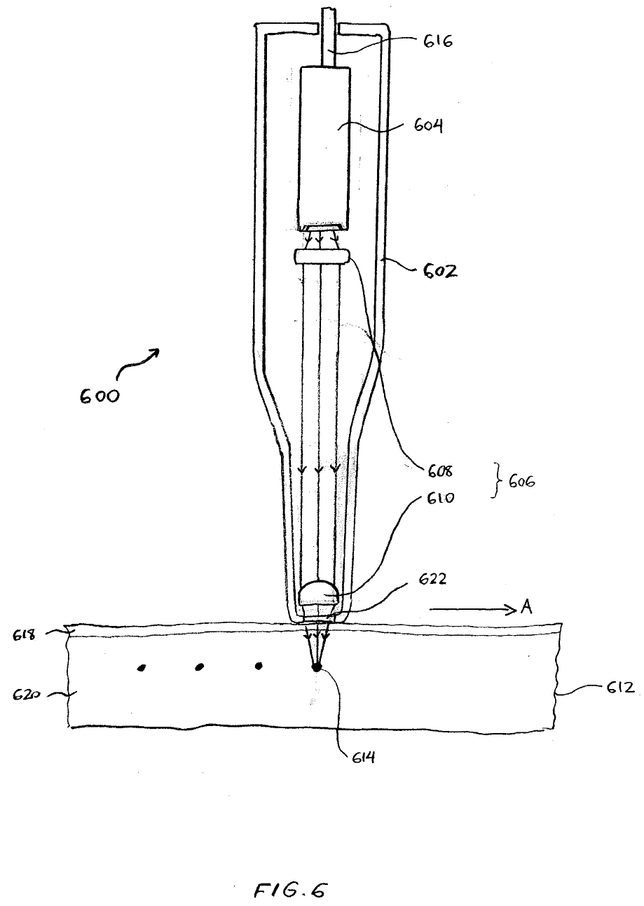

Referring next to FIG. 5, a dermatological apparatus 500 in accordance with another embodiment of the present invention is shown. The dermatological apparatus 500 includes an optical delivery system 502, which includes an optical source 504. The optical source 504 functions to provide optical energy that can be directed to a targeted portion 508 of a skin and may be implemented in a similar fashion as discussed for the optical source 104.

As illustrated in FIG. 5, the optical delivery system 502 also includes a scanning element 516 that is coupled to the optical source 504. The scanning element 516 functions to scan optical energy from the optical source 504 across the targeted portion 508. In the present embodiment, the scanning element 516 is optically coupled to the optical source 504 and scans optical energy across the targeted portion 508 such that the optical energy is directed in a pattern, such as a discontinuous pattern, to one or more treatment zones within the targeted portion 508. In particular, the scanning element 516 can scan one or more optical beams across the targeted portion 508 such that multiple treatment zones are sequentially exposed to optical energy. In the present embodiment, the scanning element 516 may be implemented, at least in part, using a scanner, such as a one-dimensional scanner or a two-dimensional scanner.

Referring to FIG. 5, the optical delivery system 502 further includes a focusing element 506 that is optically coupled to the scanning element 516. The focusing element 506 functions to direct optical energy to the targeted portion 508 by focusing the power of the optical energy to one or more treatment zones within the targeted portion 508. The focusing element 506 may be implemented in a similar fashion as discussed for the focusing element 106. It should be recognized that the focusing element 506 may be used to focus one or more optical beams to virtually any area or structure within the targeted portion 508, such as an epidermal layer 510 or a dermal layer 512 of the targeted portion 508. While the scanning element 516 and the focusing element 506 are shown separate in FIG. 5, it is contemplated that the scanning element 516 and the focusing element 506 may be implemented in a combined fashion as a scanning/focusing element.

In the present embodiment, the optical delivery system 502 additionally includes a skin deformation element 518, which, functions to deform the targeted portion 508. By way of example, the skin deformation element 518 can deform the targeted portion 508 in a substantially flat manner, a substantially concave manner, or a substantially convex manner. By thus deforming the targeted portion 508, the skin deformation element 518 provides a smoother treatment surface and allows for better accuracy and control over the delivery of optical energy to the targeted portion 508. Desirably, the skin deformation element 518 functions to apply pressure to the targeted portion 508. The application of pressure can serve to compress the targeted portion 508 and force optically absorbing interstitial fluid away from the targeted portion 508, thereby allowing a greater degree of penetration of optical energy into the targeted portion 508.

In the present embodiment, the skin deformation element 518 may be implemented, at least in part, using one or more structures, such as a skin contact element, a vacuum system, or a skin stretching element, to deform the targeted portion 508. While the focusing element 506 and the skin deformation element 518 are shown separate in FIG. 5, it is contemplated that the focusing element 506 and the skin deformation element 518 may be implemented in a combined fashion as a focusing/skin deformation element. For example, since the focusing element 506 forms a part of the dermatological apparatus 500, it would reduce the number of parts in the dermatological apparatus 500 to use the focusing element 506 for focusing as well as for skin deformation.

Referring to FIG. 5, the dermatological apparatus 500 also includes a control system 514. The control system 514 is electronically coupled to the optical delivery system 502 via any wire or wireless transmission channel and functions to control operation of the optical delivery system 502, including the optical source 504, the scanning element 516, the focusing element 506, the skin deformation element 518, or a combination thereof. By way of example, the control system 514 can control the scanning element 516 to control or adjust a pattern of optical energy that is directed to the targeted portion 508. In the present embodiment, the control system 514 may be implemented in a similar fashion as discussed for the control system 114.

As illustrated in FIG. 5, the optical delivery system 502 of the present embodiment includes a sensing element 520 that functions to detect either of, or both, movement and position of the optical delivery system 502 with respect to the targeted portion 508. In particular, the sensing element 520 can provide either of, or both, movement and position data to the control system 514 to allow substantially real time control of a pattern of optical energy that is directed to the targeted portion 508. In particular, movement data provided by the sensing element 520 can allow the control system 514 to appropriately control operation of the optical delivery system 502 to account for or compensate for movement of the optical delivery system 502 with respect to the targeted portion 508. For instance, based on such movement data, the control system 514 can control the optical source 504 or the scanning element 516 to ensure integrity and substantial uniformity of the pattern of optical energy that is directed to the targeted portion 508. In the present embodiment, the sensing element 520 may be implemented, at least in part, using a movement or position detector, such as a mechanical mouse or an optical mouse.

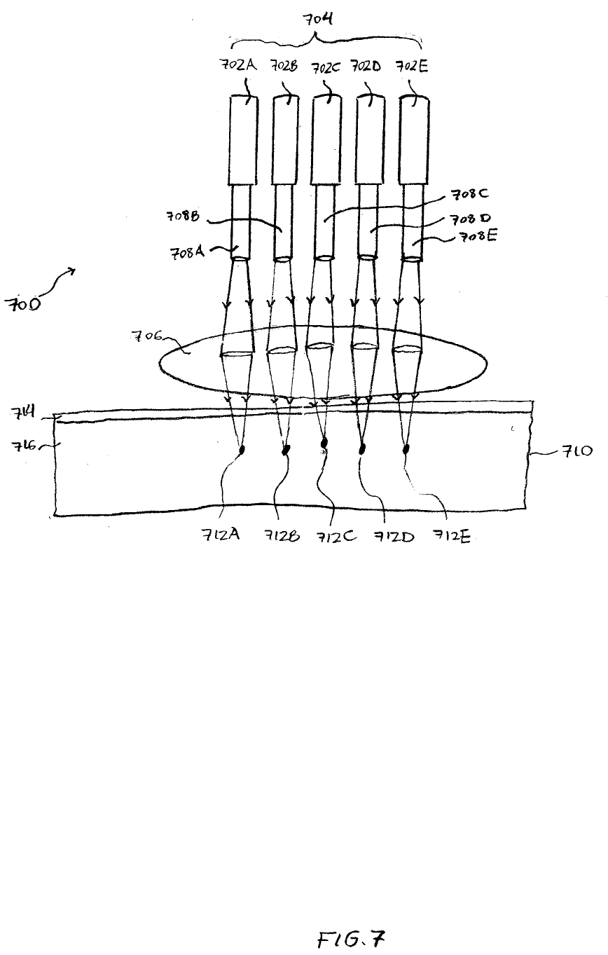

Attention next turns to FIG. 6, which illustrates an optical delivery system 600 in accordance with an embodiment of the present invention. The optical delivery system 600 includes a housing 602 sized for manipulation by a human hand. In particular, the housing 602 is sized to allow the optical delivery system 600 to be manually scanned across a targeted portion 612 of a human skin, such as along the direction of arrow A. It should be recognized that the targeted portion 612 is illustrated in FIG. 6 in a magnified form for ease of presentation.

Located within and coupled to the housing 602 are an optical source 604 and a focusing element 606. The optical source 604 can be coupled to a control system (not shown) via a cable 616. In the present embodiment, the optical source 604 is desirably an anamorphic optical source and is implemented using a diode laser, such as an infrared diode laser. More particularly, the diode laser is desirably a linear array diode laser capable of providing a substantially uniform optical beam that is expanded along a direction substantially orthogonal to arrow A, such as a direction extending out of or into the plane of FIG. 6. By manually scanning the optical delivery system 600 in conjunction with pulsed or intermittent application of optical energy, a “line pattern” of optical energy can be directed to the targeted portion 612. Also, by manually rescanning the optical delivery system 600 along a direction at an angle relative to arrow A, an “intersecting line pattern” of optical energy can be directed to the targeted portion 612.

While one diode laser is shown in FIG. 6, it is contemplated that the optical delivery system 600 may include multiple diode lasers arranged in an array, such as a one-dimensional array or a two-dimensional array. For the case of a one-dimensional array, for instance, the optical delivery system 600 can be manually scanned in conjunction with pulsed or intermittent application of optical energy such that a “dot pattern” of optical energy is directed to the targeted portion 612. It is also contemplated that the optical delivery system 600 may include a scanning element that scans one or more optical beams from the optical source 604 across the targeted portion 612. For the case of a one-dimensional scanner, for instance, the optical delivery system 600 can be manually scanned in conjunction with operation of the scanner such that a “dot pattern” or a “line pattern” of optical energy is directed to the targeted portion 612. While the optical source 604 is shown located within the housing 602, it is contemplated that the optical source 604 may be located elsewhere and may be optically coupled to the focusing element 606 via, for instance, an optical waveguide or a fiber optic cable containing one or more optical fibers.

Referring to FIG. 6, the focusing element 606 functions to direct optical energy from the optical source 604 to the targeted portion 612 via an optical window 622. Desirably, a layer of a material may be applied to the targeted portion 612 for optical contact, refractive index matching, and for comfort. In the present embodiment, the focusing element 606 includes first and second optical lens 608 and 610. Those skilled in the art will appreciate, however, that the focusing element 606 may include other optical elements (not shown) to direct optical energy to the targeted portion 612. The first optical lens 608 functions to condition and collimate an optical beam from the optical source 604. The first optical lens 608 may be implemented using, for instance, an aspheric optical lens with a substantially plano-convex cylindrical shape.

The second optical lens 610 functions to focus the power of the collimated optical beam to a treatment zone, such as treatment zone 614. In the present embodiment, the second optical lens 610 has a numerical aperture between approximately 0.15 and 1.5, such as between approximately 0.5 and 1, and may be implemented using, for instance, an optical lens with a substantially plano-convex cylindrical shape. In the present embodiment, the second optical lens 610 allows optical beams having adequate power to be focused to treatment zones within a dermal layer 620 of the targeted portion 612 while substantially avoiding damaging an epidermal layer 618 of the targeted portion 612. In particular, the optical fluence and therefore the induced temperature rise at the epidermal layer 618 can be considerably less than the optical fluence and the induced temperature rise at the focal plane deeper within the targeted portion 612, such as in the dermal layer 620. As illustrated in FIG. 6, the second optical lens 610 focuses the power of optical beams to treatment zones that are separated from one another and are relatively small or microscopic in scale along at least one dimension. Such implementation allows for greater efficacy of treatment while reducing trauma to tissue surrounding the treatment zones as well as tissue that is penetrated by the optical beams prior to reaching the treatment zones. Furthermore, such implementation reduces visible impressions of treatment because an individual treatment zone is on the same or smaller scale than the normal visible texture or constituents of the skin itself.

In the present embodiment, the treatment zones can be located up to approximately 1.5 mm below an outer surface of the skin, such as between approximately 0.15 mm and 1 mm below the outer surface. While the treatment zones are shown in the dermal layer 620 of the targeted portion 612, it is contemplated that the focusing element 606 may be used to focus optical beams to virtually any area or structure within the targeted portion 612. For instance, the focusing element 606 may be used to focus optical beams to or near the outer surface of the targeted portion 612 for a skin resurfacing treatment, such as a superficial ablative procedure. Desirably, a wavelength or range of wavelengths having high tissue absorption and low depth of penetration is used, such as between approximately 1400 mm and 14,000 nm and typically between approximately 1400 nm and 3400 nm. Tissue absorption can vary with wavelength, and, for certain applications, a wavelength or range of wavelengths is desirably chosen for which tissue absorption is highest, such as at or near 1450 nm and above 2500 nm. Skin is approximately 70 percent water, and water absorption curves can be a useful reference for locating a desirable wavelength or range of wavelengths for treatment. For certain applications, it is contemplated that two or more different wavelengths or wavelength ranges can be used, such as a first wavelength or wavelength range having low tissue absorption and high depth of penetration and a second wavelength or wavelength range having high tissue absorption and low depth of penetration. By way of example, an optical beam having the first wavelength or wavelength range can be directed to the targeted portion 612 to achieve a pre-heating effect as well as produce coagulation of tissue down to the dermal layer 620 of the targeted portion 612, and an optical beam having the second wavelength or wavelength range can be directed to the targeted portion 612 to achieve superficial ablation of the epidermal layer 618.

For a skin resurfacing treatment, one or more holes may be formed across the outer surface of the targeted portion 612 at locations that are exposed to optical beams. Multiple holes may be formed with depths between approximately 10 μm and 1000 μm, such as between approximately 10 μm and 300 μm. For certain applications, holes are desirably formed with sizes between approximately 10 μm and 1000 μm, such as between approximately 50 μm and 500 μm. Multiple holes can be separated from one another so as to form discrete holes. Alternatively, or in conjunction, multiple holes can intersect or overlap one another. Depending on the particular treatment level and wavelength used, it is contemplated that one or more zones of thermally denatured tissue may be formed instead of, or in conjunction with, one or more holes, which denatured tissue may be subsequently sloughed off or absorbed by the body to achieve a similar skin resurfacing effect as discussed above. In particular, the desired result is the replacement of the denatured tissue by fresh tissue and the associated stimulation of new collagen and other beneficial proteins that improve the quality, appearance, and youthful character of the skin.

While not shown in FIG. 6, it is contemplated that the optical delivery system 600 may include a sensing element that can function to detect either of, or both, movement and position of the optical delivery system 600 with respect to the targeted portion 612. For instance, the sensing element may detect movement of the optical delivery system 600 as it is manually scanned across the targeted portion 612 to allow optical energy to be directed in a controlled fashion to the targeted portion 612. In particular, movement data provided by the sensing element may allow an appropriately programmed control system to alter one or more optical parameters, such as timing, to ensure integrity and substantial uniformity of the pattern of optical energy that is directed to the targeted portion 612.

Referring next to FIG. 7, an optical delivery system 700 in accordance with another embodiment of the present invention is illustrated. The optical delivery system 700 includes an optical source 704 and a focusing element 706 that is optically coupled to the optical source 704. In the present embodiment, the optical source 704 includes multiple light sources 702A, 702B, 702C, 702D, and 702E that are arranged in an array. The light sources 702A-702E may include one particular type of laser light source or two or more different types of laser light sources. While five light sources 702A-702E are shown in FIG. 7, it is contemplated that more or less light sources can be used depending on the specific application.

In the present embodiment, the light sources 702A-702E are connected, on a one-by-one basis, to optical pathways 708A, 708B, 708C, 708D, and 708E, as illustrated in FIG. 7. For such implementation, each of the light sources 702A-702E is capable of delivering an optical beam through it own optical pathway to a targeted portion 710 of a human skin. Since the light sources 702A-702E are connected, on a one-by-one basis, to the optical pathways 708A-708E, a pattern of optical energy can be provided and delivered to the targeted portion 710. To accomplish such a pattern, a control system (not shown) can be electronically coupled to the light sources 702A-702E to select and activate one or more of the light sources 702A-702E as well as control a variety of optical parameters associated with an activated light source. In the present embodiment, the optical pathways 708A-708E are desirably optical fibers with diameters ranging from single mode fiber diameters to approximately 1 mm. However, it is contemplated that the optical pathways 708A-708E are not limited to optical fibers and, for example, could be any type of optical waveguide. It is also contemplated that optical elements, such as mirrors or optical lenses, may be employed within the context of the present embodiment to provide the functionality of the optical pathways 708A-708E.

Referring to FIG. 7, the focusing element 706 functions to focus the power of optical beams delivered via the optical pathways 708A-708E to multiple treatment zones 712A, 712B, 712C, 712D, and 712E within the targeted portion 710. In the present embodiment, the treatment zones 712A-712E desirably have sizes between approximately 10 μm and 1000 μm, such as between approximately 50 μm and 500 μm, and are separated from one another so as to form discrete treatment zones. The treatment zones 712A-712E can be located up to approximately 1.5 mm below an outer surface of the skin, such as between approximately 0.15 mm and 1 mm below the outer surface. For certain applications, different treatment zones can be located at different depths below the outer surface of the skin by, for example, arranging the optical pathways 708A-708E at different positions relative to the focusing element 706. While the treatment zones 712A-712E are shown in a dermal layer 716 of the targeted portion 710, it is contemplated that the focusing element 706 may be used to focus one or more optical beams to virtually any area or structure within the targeted portion 710, such as an epidermal layer 714 of the targeted portion 710. It is contemplated that the focusing element 706 may be used to focus optical beams to or near the outer surface of the targeted portion 710 for a skin resurfacing treatment, such as a superficial ablative procedure, in a similar manner as discussed in connection with FIG. 6.

While FIG. 7 illustrates the focusing element 706 as including one optical lens, those skilled in the art will appreciate, however, that the focusing element 706 may include other optical elements (not shown) to direct optical energy to the targeted portion 710. For instance, it is contemplated that the focusing element 706 may include two or more optical lenses. Different optical lens sizes may be used ranging, for example, from a 2-mm diameter optical lens to a 2-inch diameter optical lens. For certain applications, the focusing element 706 could be extended with individual optical elements (not shown) for each of the optical pathways 708A-708E.

It should be recognized that the specific embodiments of the present invention discussed above are provided by way of example, and various other embodiments are encompassed by the present invention.

For instance, some embodiments of a dermatological apparatus may include a viewing system, a recording system, a displaying system, or a combination thereof. The viewing system can allow a user to view a targeted portion of a skin and may be implemented, for instance, using an observation window coupled to or included within an optical delivery system. The recording system can function to record reflected light from the targeted portion and may be implemented, for instance, using a camera or Charge Coupled Device (“CCD”) imager to record reflections in the infrared or visible spectrum. Once infrared or visible reflections are recorded, the recorded reflections can be processed by a control system and displayed as infrared or visible data using the displaying system. The displaying system may be implemented, for instance, using a computer screen, flat panel display, personal digital assistant, or wireless communication device that allows display of data.

Some embodiments of a dermatological apparatus may include a sensing element that can function to provide data to an appropriately programmed control system to allow substantially real time targeting of a pattern of optical energy to treat skin. In particular, it is contemplated that such embodiments can automatically treat skin using color or other detectable optical properties to distinguish between normal skin and skin which requires treatment, thereby sparing normal tissue from unnecessary trauma while treating microscopically adjacent tissue which requires treatment. The sensing element may be implemented, for instance, using color-discriminating detectors as described in U.S. Pat. No. 5,531,740 to Black, entitled “Automatic Color-Activated Scanning Treatment of Dermatological Conditions by Laser,” the disclosure of which is incorporated herein by reference in its entirety.

As another example, some embodiments of a dermatological apparatus may include a cooling system. The cooling system can function to dynamically or statically control the temperature of a targeted portion of a skin prior to, during, or after treatment and may be implemented, for instance, using a fluid delivery apparatus or a cold skin contact element.

As a yet another example, some embodiments of the present invention relate to the treatment of a wide variety of biological tissues using patterns of optical energy. In particular biological tissues that have an epithelial protective layer corresponding to an epidermal layer of skin also may be treated in a similar manner as discussed herein. For instance, patterns of optical energy may be applied to the soft palate for treatment of snoring and sleep apnea.

The following example describes specific aspects of the present invention to illustrate and provide a description of the present invention for those of ordinary skill in the art. The example should not be construed as limiting the present invention, as the example merely provides specific methodology useful in understanding and practicing the present invention.

### EXAMPLE

In vitro skin (sample size=4 mm×6 mm) was placed next to a glass plate with an anti-reflective coating and compressed slightly with a small weight. Optical energy (wavelength=1500 nm; pulse duration=10 ms; and pulse power=1000 mW) from a laser light source was delivered using an optical fiber and then focused through the glass plate and within the skin using a beam collimator and a focusing objective (numerical aperture=0.53). The depth of a treatment zone that was exposed to optical energy could be varied between approximately 500 μm to 700 μm below an outer surface of the skin by adjusting the distance between the focusing objective and the glass plate. A transparent lotion was used as an index matching material between the glass plate and the skin. This lotion also helped keep the skin moist and improved conduction of excess thermal energy away from a treatment zone. A single laser pulse was directed to each treatment zone, and, in this fashion, various treatment zones within the skin were exposed to optical energy. The treatment zones were distributed at intersection points of a rectangular grid and were spaced apart by a distance of approximately 500 μm. The treatment zones were generally elongated and had widths of approximately 200 μm.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations are considered to be within the scope and spirit of the present invention as defined by the following claims and their legal equivalents.

What is claimed is:

1. A dermatological apparatus comprising:
a plurality of light source and optical pathway connections, wherein each light source in said plurality of light source and optical pathway connections is capable of delivering an optical beam through its connected optical pathway to a targeted portion of a human skin;
a control system to select and control said light sources to deliver a plurality of optical beams in a discontinuous pattern; and
a focusing element to focus the power of said delivered optical beams to a plurality of discrete treatment zones that are located up to 1.5 mm underneath an outer surface of said targeted portion, said discrete treatment zones having sizes in the range of 10 μm to 1000 μm.

2. The dermatological apparatus of claim 1, wherein said discrete treatment zones have sizes in the range of 50 μm to 500 μm.

3. The dermatological apparatus of claim 1, wherein said discrete treatment zones are located in a dermal layer of said targeted portion.

4. The dermatological apparatus of claim 3, wherein said focusing element focuses the power of said delivered optical beams to said discrete treatment zones while substantially avoiding damaging an epidermal layer of said targeted portion.

5. A dermatological apparatus comprising:
a plurality of light source and optical pathway connections, wherein each light source in said plurality of light source and optical pathway connections is capable of delivering an optical beam through its connected optical pathway to an outer portion of a human skin;
a control system to select and control said light sources to deliver a plurality of optical beams in a discontinuous pattern; and
a focusing element to focus the power of said delivered optical beams to said outer portion to form a plurality of discrete holes distributed across said outer portion, said discrete holes having sizes in the range of 10 μm to 1000 μm.

6. The dermatological apparatus of claim 5, wherein said discrete holes have sizes in the range of 50 μm to 500 μm.

7. The dermatological apparatus of claim 5, wherein said discrete holes have depths in the range of 10 μm to 1000 μm.

8. The dermatological apparatus of claim 5, wherein said discrete holes are distributed across said outer portion with a fill factor in the range of 0.1 to 0.5.

9. A dermatological apparatus comprising:
an optical delivery system, said optical delivery system including
an optical source, said optical source being configured to provide optical energy having a wavelength in the range of 400 nm to 20,000 nm; and
a focusing element optically coupled to said optical source, said focusing element being configured to direct said optical energy in a discontinuous pattern to a targeted portion of a skin.

10. The dermatological apparatus of claim 9, wherein said optical delivery system further includes a housing sized for manipulation by a human hand, said optical source and said focusing element being located within said housing.

11. The dermatological apparatus of claim 9, wherein said optical source includes a plurality of laser light sources.

12. The dermatological apparatus of claim 9, wherein said focusing element is configured to direct said optical energy in said discontinuous pattern to said targeted portion such that a plurality of discrete treatment zones within said targeted portion are exposed to said optical energy.

13. The dermatological apparatus of claim 12, wherein said discrete treatment zones have sizes in the range of 10 μm to 1000 μm.

14. The dermatological apparatus of claim 12, wherein said discrete treatment zones are located in at least one of an epidermal layer and a dermal layer of said targeted portion.

15. The dermatological apparatus of claim 9, wherein said focusing element is configured to direct said optical energy to said targeted portion in one of a dot pattern and a line pattern.

16. The dermatological apparatus of claim 9, wherein said focusing element is configured to direct said optical energy to said targeted portion in the form of a plurality of optical beams.

17. The dermatological apparatus of claim 9, wherein said optical delivery system further includes a scanning element optically coupled to said optical source and to said focusing element, said scanning element being configured to scan said optical energy across said targeted portion.

18. A dermatological apparatus comprising:
an optical delivery system, said optical delivery system including
an optical source; and
a focusing element optically coupled to said optical source, said focusing element being configured to direct optical energy from said optical source to a targeted portion of a skin, said focusing element including an optical lens having a numerical aperture in the range of 0.15 to 1.5, said optical lens being configured to focus said optical energy to a dermal layer of said targeted portion.

19. The dermatological apparatus of claim 18, further comprising:
a control system electronically coupled to said optical delivery system, said control system being configured to control said optical delivery system.

20. The dermatological apparatus of claim 18, wherein said optical source includes at least one laser light source.

21. The dermatological apparatus of claim 18, wherein said optical source is configured to provide said optical energy having a wavelength in the range of 400 nm to 20,000 nm.

22. The dermatological apparatus of claim 18, wherein said optical lens is configured to focus said optical energy to a treatment zone within said dermal layer of said targeted portion while substantially avoiding damaging an epidermal layer of said targeted portion.

23. The dermatological apparatus of claim 22, wherein said treatment zone has a size in the range of 10 μm to 1000 μm.

24. The dermatological apparatus of claim 22, wherein said treatment zone is located in the range of 0.15 mm to 1 mm underneath an outer surface of said targeted portion.

25. The dermatological apparatus of claim 22, wherein said treatment zone is substantially circular.

26. The dermatological apparatus of claim 22, wherein said treatment zone is elongated.

27. A dermatological apparatus comprising:
a housing sized for manipulation by a human hand;
an optical source located within said housing, said optical source being configured to provide optical energy; and
a focusing element coupled to said housing, said focusing element being configured to direct said optical energy to a targeted portion of a skin such that a plurality of treatment zones within said targeted portion are exposed to said optical energy, said treatment zones being separated from one another within said targeted portion.

28. The dermatological apparatus of claim 27, further comprising:
a control system electronically coupled to said optical source, said control system being configured to control said optical source to provide said optical energy.

29. The dermatological apparatus of claim 27, further comprising:
a skin deformation element coupled to said housing, said skin deformation element being configured to deform said targeted portion.

30. The dermatological apparatus of claim 27, wherein said optical source is configured to provide said optical energy having a wavelength in the range of 600 nm to 4000 nm.

31. The dermatological apparatus of claim-27, wherein said optical source includes at least one diode laser.

32. The dermatological apparatus of claim 27, wherein said focusing element is configured to direct said optical energy to said targeted portion such that said treatment zones are substantially simultaneously exposed to said optical energy.

33. The dermatological apparatus of claim 27, wherein said focusing element is configured to direct said optical energy to said targeted portion such that said treatment zones are sequentially exposed to said optical energy.

34. The dermatological apparatus of claim 27, wherein said focusing element includes at least one optical lens, said optical lens being configured to focus said optical energy up to 1.5 mm underneath an outer surface of said targeted portion.

35. The dermatological apparatus of claim 27, wherein said focusing element includes at least one cylindrical lens having a numerical aperture in the range of 0.15 to 1.5.

36. The dermatological apparatus of claim 27, wherein said treatment zones are distributed substantially uniformly across said targeted portion.

37. The dermatological apparatus of claim 27, wherein said treatment zones have sizes in the range of 50 μm to 500 μm.

38. A dermatological apparatus comprising:
an optical delivery system, said optical delivery system being configured to direct optical energy in a pattern to a targeted portion of a skin such that a plurality of discrete treatment zones within said targeted portion are exposed to said optical energy, said discrete treatment zones having sizes in the range of 10 μm to 1000 μm.

39. The dermatological apparatus of claim 38, wherein said optical delivery system includes a focusing element, said focusing element being configured to direct said optical energy up to 1.5 mm underneath an outer surface of said targeted portion.

40. The dermatological apparatus of claim 39, wherein said optical delivery system further includes an optical source optically coupled to said focusing element, said optical source being configured to provide said optical energy having a wavelength in the range of 400 nm to 20,000 mm.

41. A method of treating a human skin, comprising:
providing optical energy, said optical energy having optical parameters to produce a dermatological effect for a targeted portion of said human skin; and
directing said optical energy to said targeted portion such that a plurality of discrete treatment zones within said targeted portion are substantially simultaneously exposed to said optical energy.

42. The method of claim 41, wherein two adjacent discrete treatment zones of said plurality of discrete treatment zones are separated by a distance in the range of 100 μm to 1000 μm.

43. The method of claim 41, wherein directing said optical energy to said targeted portion includes focusing said optical energy to a dermal layer of said targeted portion while substantially avoiding damaging an epidermal layer of said targeted portion.

44. A method of treating a human skin, comprising:
providing optical energy; and
directing said optical energy to an outer portion of said human skin to form a plurality of discrete holes distributed across said outer portion, said discrete holes having sizes in the range of 10 μm to 1000 μm.

45. The method of claim 44, wherein said optical energy has a wavelength in the range of 1400 nm to 14,000 nm.

46. The method of claim 44, wherein said discrete holes have depths in the range of 10 lm to 1000 μm.

47. The method of claim 44, wherein said discrete holes are distributed across said outer portion with a fill factor in the range of 0.05 to 0.95.

48. The method of claim 44, wherein said discrete holes are distributed across said outer portion with a fill factor in the range of 0.1 to 0.5.

49. The method of claim 44, wherein directing said optical energy to said outer portion includes scanning said optical energy across said outer portion.
